(19) United States
(12) Patent Application Publication
(10) Pub. No.: US 2015/0335506 A9
(43) Pub. Date: Nov. 26, 2015

(54) Sleep Cycle Bed

(71) Applicant: Boyd Thomas Kildey, Brisbane (AU)

(72) Inventor: Boyd Thomas Kildey, Brisbane (AU)

(21) Appl. No.: 13/970,354

(22) Filed: Aug. 19, 2013

Related U.S. Application Data
(60) Provisional application No. 61/699,267, filed on Sep. 10, 2012.

(30) Foreign Application Priority Data
Sep. 11, 2012 (AU) 2012903949
Aug. 14, 2013 (AU) 2013216618

(51) Int. Cl. A61G 7/018; A61M 21/00

(52) U.S. Cl. CPC A61G 7/018; A61M 21/00

(57) ABSTRACT

The Invention is a decision making process and method to govern the combination of existing prior art associated with Pressure Bed Systems and sleep analysis tools. The invention creates a unique product that uses the biological signals of a User to generate an automated response to control a bed or mattress structure that is constructed with the relevant equipment to change the relative firmness or softness (to be described as ‘pressure’) of the bed or mattress. The intention of the automation is to better the quality of the User's sleep through the influencing of the User's bed or mattress and the assisted waking of the User at a User defined time through the same mechanism.

Fields of Invention
a) Beds and mattress
b) Sleep aids
c) Sleep analysis

### DESCRIPTION OF THE INVENTION

The Invention is the process and method for the combination of existing Sleep Cycle Monitoring technology and Pressure Bed Systems to create a unique product in the form of an automated Pressure Bed System.

The automated Pressure Bed System retains existing Pressure Bed System characteristics . and technology with the addition of process that:
a) Adapt the Pressure Bed System to a User's Sleep Cycle;
b) Actively advance the quality of a User's sleep;
c) Actively aid in the awakening process of a User; &
d) Connect wirelessly the Pressure Bed System with extrinsic technology for the input and output of commands and data.

The Invention is capable of:
a) Mass production;
b) Manual or automated control of the Pressure Bed System through a remote control input (wirelessly and/or tethered);
c) Monitoring of biological signals of the User to determine the Sleep Cycle of the User;
d) Automatically adapting the Pressure Bed System to suit the Sleep Cycle of the User;
e) Automatically adapt the Pressure Bed System to control the quality of the User's sleep;
f) Recording biological signals and Sleep Cycle information to provide a Sleep Cycle report;
g) Connecting (wirelessly or tethered) to smart phone or computer software to graphically present Sleep Cycle data;
h) Utilising historical data within an algorithm to make decisions;
i) Utilising historical data to influence the algorithm and improve the decision making;
j) Receiving waking alarm information (wirelessly or tethered) from a smart phone or computer to automatically adapt the Pressure Bed System to optimise waking environment for the User; &
k) Selecting a time according to a decision making algorithm to begin a waking procedure.

### DEFINITIONS & TERMS

Alternative User Sleep Cycle Models:

Biological Signals: means the User's movements, heartbeat, temperature, breathing rate or any other biological output that can be measured for the purpose of determining a User's Sleep Cycle.

Ensemble: a bed structure generally made up of a bed base and mattress, where the bed base is designed to work functionally with the mattress.

Pressure: is the measure of relative firmness or softness which can be adapted by command.

Pressure Bed System: includes any bed or mattress that is capable of modifying the pressure of the bed or mattress via an input command. The term “Air Bed Systems” is used as general industry for most of these bed types; however it is irrelevant to this invention whether a change in the pressure in the bed or mattress is related or directly achieved through the use of air, springs, fluid or any other method or material.

Sleep Cycle: the varying patterns or stages of human sleep, fluctuating between what is generally described as wakefulness to deep sleep.

Sleep Cycle Monitoring: the recording of biological data for the specific purpose of determining the varying patterns or stages of a User's sleep, fluctuating between what is generally described as wakefulness to deep sleep.

Smart Device: includes the genre of mobile devices that are at the time of drafting this document described as smart phones and tablets, but also includes personal computing devices that are capable of interaction with other devices through either wired or wireless communication. It is envisaged that this includes devices such as iPhones, iPads, Google Android based phones, Microsoft Windows mobile based phones, laptops computers and networked connected personal computers etc.

User: a person who is making use of the Pressure Bed System and its integrated technology.

User Sleep Cycle Model: A set of parameters (which are capable of amendment) that currently represent the characteristics of a user's various sleep patterns.

### BACKGROUND OF THE INVENTION (PRIOR ART)

### Air Bed System

The below description is of one form of Air Bed System and consequently one form of bed or mattress structure that can change the relative firmness or softness of the bed or mattress at command. The description of this type of Air Bed System is included merely to provide context to the latter detailed description of the Invention, however the Invention should be noted to apply to any Pressure Bed System as defined in the definition section of this document.

Air Bed Systems, such as the one described in U.S. Pat. No. 5,904,172 which is incorporated merely by reference generally allows a User to select a desired pressure for each air bladder within the mattress.

In operation, upon selecting the desired pressure, a signal is sent to a pump and valve assembly in order to inflate or deflate the air bladders as necessary to achieve the desired pressure within the air bladders.

The Air Bed System generally comprises a bladder pneumatically connected to an electric pump. The bladder is surrounded by a soft border and encased by mattress ticking.

The electric pump includes a remote control (tethered or wireless) which allows a User to inflate or deflate the bladder.

The manual operated remote control electronically communicates with a circuit board controlling the pump and monitoring the pressure in the bladder.

A control box or switch box with a programmable timing circuit operates the pump and associated relief valves such that the air pressure in the bladder changes in coordination with the instructions from the remote control

The Air Bed System will monitor and the pressure through various techniques and inflate or deflate the air bladders as necessary to ensure the maintenance of the desired pressure.

Air Bed Systems have become increasingly popular internationally due to the adaptive nature of the sleeping environment. Similar larger ensembles are sold on their attractiveness to couples who would prefer a different feeling ensemble as the Air Bed Systems can be used to have differing pressures on each side of the ensemble.

Current Air Bed Systems maintain to a reasonable accuracy the pressure within the air bladder to the User's predefined setting.

A User must actively change the desired pressure for the Air Bed System to calibrate the bladder to a different pressure.

### Sleep Cycle Monitoring

Sleep Cycle Monitoring, such as described in U.S. Pat. No. 7,608,041 which is incorporated merely by reference generally allows a User to monitor and discriminate a Sleep Cycle of a User and selectively compute an optimum waking time of the User.

The monitoring system generally comprises:
a) A means for setting a measuring time, &
b) A means for measuring a biological signal (movement in this instance) of the User.

The computational system generally comprises:
a) Computing means for providing variation indexes representing variation of a biological signal in denoting a tendency of increment in a time series from a starting time of the measuring to a time when the biological signal is obtained with second variation amount denoting a standard variation of the biological signal during said the following measuring time;
b) A sleep index operating means for providing sleep indexes allowing a Sleep Cycle and all other Sleep Cycles to be discriminated from one another on the basis of said variation indexes be compared to predetermined thresholds; and
c) A computing means for calculating an optimal awakening time based on current Sleep Cycles and awakening the User to maximize Sleep Cycles.

The wakening system generally comprises:
a) A means for generating an awakening stimulus to be given to the User,
b) A means for setting an awakening time;
c) A means for controlling operation of said awakening stimulus.
d) A means for setting a reference value which can be regarded as said biological signal at a stably resting and awakening state on the basis of the biological signal at initial stage of the measurement, and means for presuming a sleep-on set time when the biological signal is not within the said predetermined threshold set on the basis of said reference value.

e) A means for judging a state of re-sleeping on the basis of a biological signal measured after an actuation of said awakening stimulus generating means and actuating again the awakening stimulus generating means.

### PATENT BACKGROUND

1. U.S. Pat. No. 6,686,711 B2 “Air Mattress Control System and Method” Feb. 3, 2004

2. U.S. Pat. No. 4,394,784 “Air Bed with Firmness Control” Jul. 26, 1983

3. U.S. Pat. No. 5,903,941 “Air Control System for an Air Bed” May 18, 1999

4. U.S. Pat. No. 7,608,041 B2 “Monitoring and Control of Sleep Cycles” Oct. 27, 2009

5. US 2005/0190065 A1 “Natural Alarm Clock” Sep. 1, 2005

6. U.S. Pat. No. 5,928,133 “User Responsive Sleep Monitoring and Awakening Device” Jul. 27, 1999

7. US 2008/0191885 “Easy wake Device” Aug. 14, 2008

### BRIEF DESCRIPTION OF THE DRAWINGS

FIG. 1 is a functional block diagram illustrating the steps in the general operation of the Sleep Cycle Bed.

FIG. 2 is a block diagram illustrating the decision-making processes associated with the adjustment of Pressure.

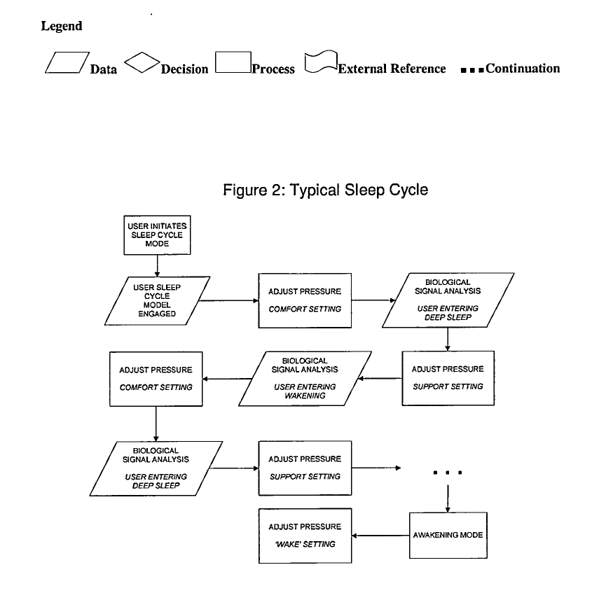

FIG. 3 is a block diagram illustrating the steps in the waking of the User

FIG. 4 is a block diagram illustrating the steps associated with data monitoring.

FIG. 5 are a block diagram illustrating the steps associated with data analytics.

FIG. 6 is a perspective view of a possible embodiment of the components of the Sleep Cycle Bed.

FIG. 7 is an elevation view of a possible embodiment of the components of the Sleep Cycle Bed.

### POSSIBLE EMBODIMENT

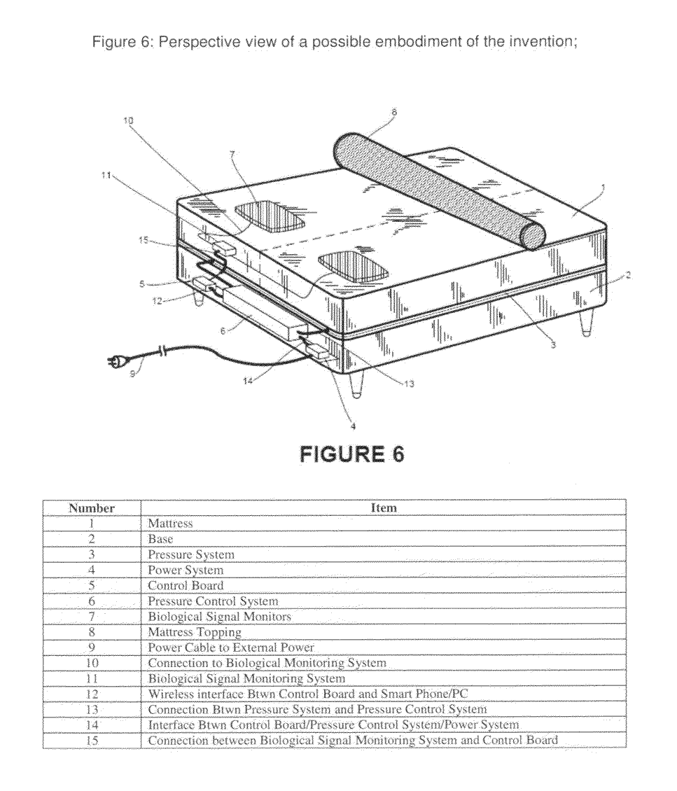

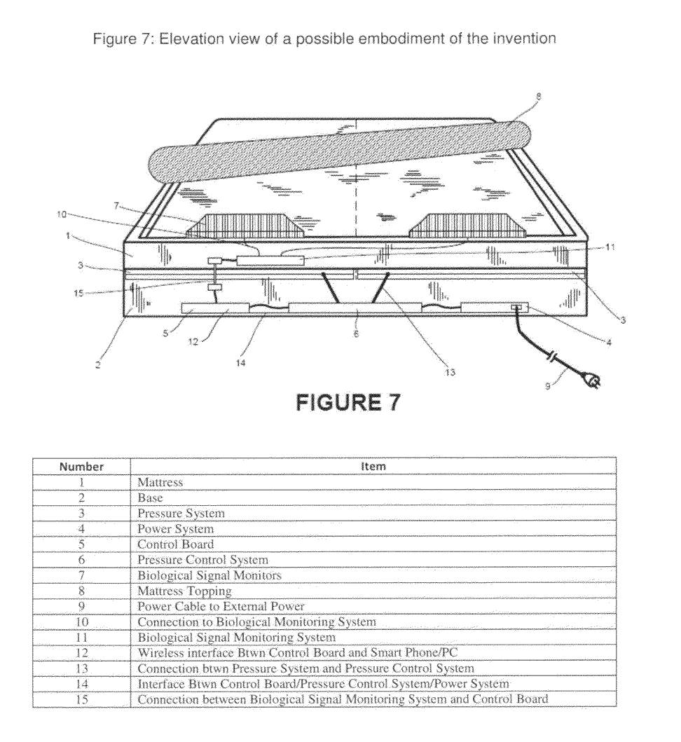

In FIG. 6 and FIG. 7 the Sleep Cycle Bed is depicted in an ensemble consisting of a mattress 1 and base 2 with all major components contained within. Within the base 2 is included the Pressure Control System 6, Control Board 5, Power System 4 and Wireless Connection 12. Within the mattress are the Biological Signal Monitors 7 and Biological Signal Monitoring System 11.

The Power System 4 provides power to all systems within the base 2 and the mattress 1. The Power System 4 connects externally to a power source via a power cable 9. The systems within the base 2 are connected to and interface with one another via series of cabling 14 capable of transmitting both power and data. Information from the Biological Signal Monitoring System 11 is transferred via cabling 15 capable of transmitting both power and data to the Control Board 5.

The Pressure System 3 is controlled by the Pressure Control System 6. It is connected via cabling 13 to transmit adjustments in pressure. In the case of an Air Bed System for the purposes of a pressure control, the Pressure Control System 6 will include a pump and tubing to transmit determined volumes of air. The Pressure System 3 is a bladder the can be filled with volumes of air to adjust the pressure of the bed.

The mattress 1 encompasses ordinary mattress features including a mattress topper 8 to provide for increased conform, support features and edging to aid with the rigidity of the mattress' 1 sides. Below the mattress topper 8, the Biological Signal Monitors 7 are situated to record the User's biological signals in the most effective manner. The Biological Signal Monitors 7 are connected via cabling 10 to the Biological Signal Monitoring System 11.

The systems of the Sleep Cycle Bed are located with the bed frame. A remote control may be connected wirelessly or via wired connection to the Control Board 5. The remote control will contain controls for the manual adjustment of the pressure, the engagement/disengagement of the automation mode, the engagement/disengagement of the waking mode and the engagement/disengagement of the connection to a Smart Device.

### Alternate Embodiments

This claim of art covers the inclusion of the process of the analysis of biological signals for the purposes of adjusting the pressure of the bed

### Biological Signal Monitors

It is envisaged that monitors within a Sleep Cycle Bed can monitor any biological signal that is capable of contributing to the determination of the User's state of sleep.

FIG. 6 shows the Biological Signal Monitors as located in a single location within the mattress. This representation is done merely for information purposes, as dependent on the biological signal sought to be monitored the placement may vary anywhere within the ensemble.

Interaction between System Control Board, Biological Monitoring Board Power & Pressure Systems

The internal control and analytical elements of the Sleep Cycle Bed may combine into an individual set of circuitry or may be kept separate. How this element of the art is constructed is not a claim of this art.

### Ensemble

FIGS. 6 & 7 exhibits the Sleep Cycle Bed in an ensemble arrangement. It is considered that any embodiment of the processes of the Sleep Cycle Bed, whether contained within an ensemble or a mattress utilising a bed constructed of a third party frame and mattress, is considered to be entirely within the contemplation of this art.

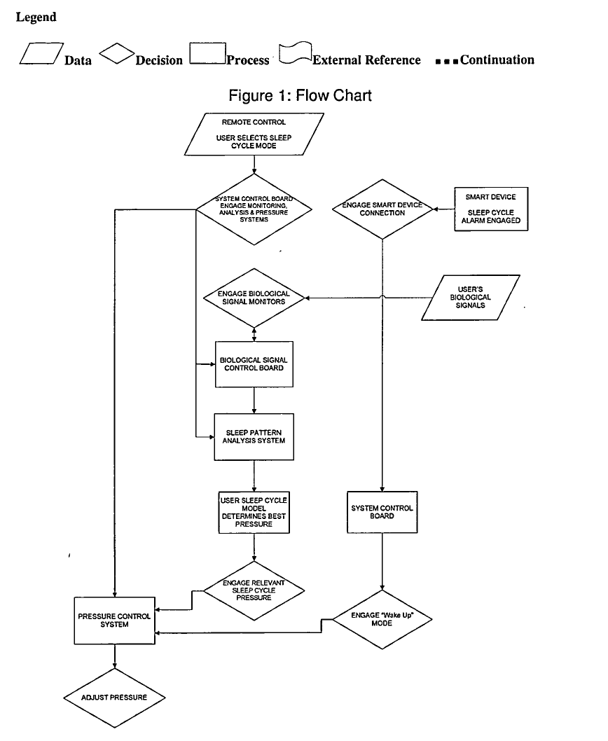

1: Automation of the Pressure Bed System
It is a claim of this Invention that it is the first to introduce a process where a User's biological signals are a stimulus for the Pressure Bed System to adjust pressure automatically during the User's sleep.
a) In this Invention the User at the outset of the sleep selects an automation setting, this setting institutes the processes as documented in FIGS. 1 & 2 which requires no further conscious input from the User to take effect.
b) Active control is not required throughout the User's sleep, as the Invention captures Biological Signals as inputs to determine the User's Sleep Cycle and adjusts the Pressure Bed System to achieve the appropriate pressure.
c) The intention of the automation is to improve the quality of the User's sleep by regulating the periods and quality of deep sleep.
d) Active outputs will be through an automated adjustment of pressure within the Pressure Bed System. The control of which will utilize existing technologies to measure and control pressure.
e) Existing Pressure Bed Systems provide Users with the ability to control their comfort and support levels at the outset of a User's sleep or manually again during the ‘sleep’ period.
f) The existing technologies discussed within the Prior Art section individually provide results within their distinct fields of sleep related products; however none provide active and varied outputs throughout a User's sleep.
g) In contrast to the Invention the existing prior art in this field does not work actively and automatically during the User's Sleep Cycle to increase the quality and comfort of the User's sleep.

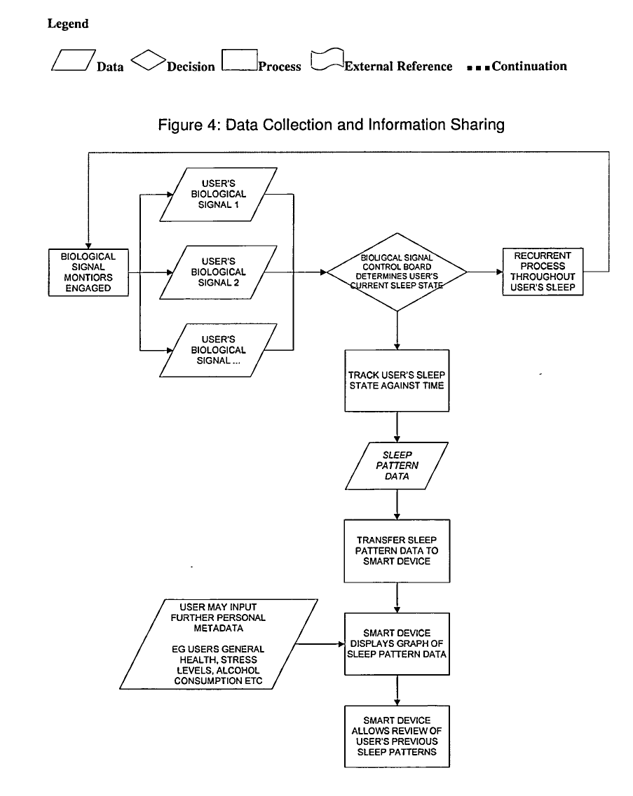

2: Data Monitoring
It is a claim of this Invention that it introduces the first process for a bed to record a User's Sleep Cycle data and for display on a Smart Device,
a) Sleep Cycle data and the representation of patterns (FIG. 4) provide Users reference material for the benefit of the User at a later date.
b) Users will have the ability to provide further metadata in relation to any individual sleep that will allow for greater efficiency in the categorization of a sleep pattern and also greater context for any review of past patterns. Metadata in this instance includes characteristics of a User's life, recent past, general health or other matter that may have an influence on a User's general sleep. For example a User's intake of alcohol, current stresses level, recent activity level, overall health or any current pain.

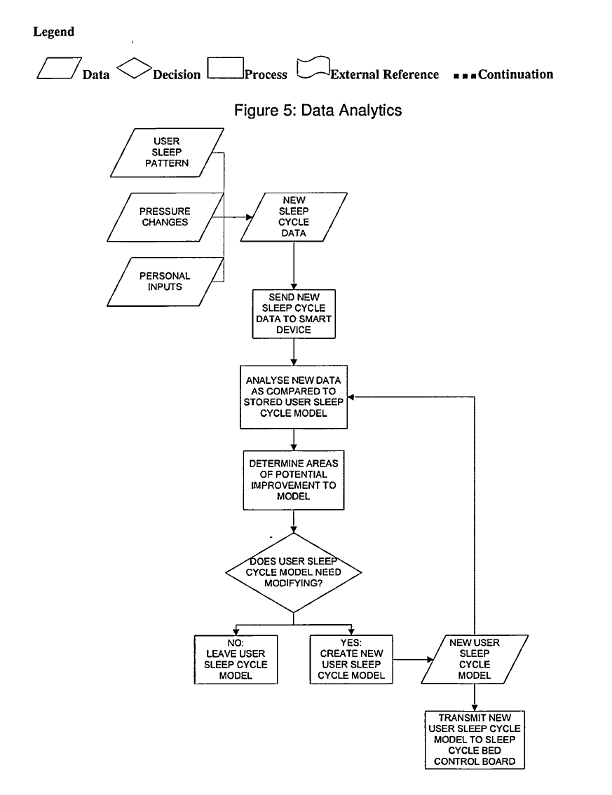

3: Data Analytics (Continual Learning)
It is a claim of this art that it includes the first process for the continual ‘learning’ (as described at FIG. 5) and adaption of the automation as described in claim 1.
a) The Sleep Cycle data as described in claim 2 is compared and analyzed against a standard automation model to determine if the appropriate pressure is being delivered to maximize the quality of the User's sleep.
b) The Smart Device which the User elects to use to communicate with the Invention will determine a User Sleep Cycle Model through evaluating the effectiveness of pressure changes in maximizing the quality of a User's sleep.
c) The metadata provided by the User will also be utilized to automatically create Alternative User Sleep Cycle Models.
d) The Invention includes a process whereby the updated User Sleep Cycle Model and Alternative Sleep Cycle Models are transferred to the System Control Board via the interface with the Smart Device as described in claim 5.

4: Data Analytics (Modification of Response)
It is a claim of this art that it includes the first process for the adaption of the automation as described in claim 1
e) The Sleep Cycle data as described in claim 2 is compared and analyzed against a standard automation model to determine if the appropriate pressure is being delivered to maximize the quality of the User's sleep.
f) The Smart Device which the User elects to use to communicate with the Invention will determine a User Sleep Cycle Model through evaluating the effectiveness of pressure changes in maximizing the quality of a User's sleep.
g) The metadata provided by the User will also be utilized to automatically create Alternative User Sleep Cycle Models.
h) The Invention includes a process whereby the updated User Sleep Cycle Model and Alternative Sleep Cycle Models are transferred to the System Control Board via the interface with the Smart Device as described in claim 5.

5: External Connection
It is a claim of this art that it introduces the first process for a bed to utilize wired and or wireless technology to interface with a Smart Device to control the mechanical and electrical functions of a bed for the purpose of actively and automatically maximizing the quality of User's sleep.
a) Connectivity will be provided through any form of wired or wireless transmission that allows the movement of data in both directions between the bed and a Smart Device.
b) The art includes a process for the delivery of distinct packets of information necessary for the Smart Device to complete actions under claims 2, 3 & 4.
c) It is a claim of this art that it includes a process for the control of the functions of the bed through the receipt of distinct packets of data received from a Smart device.

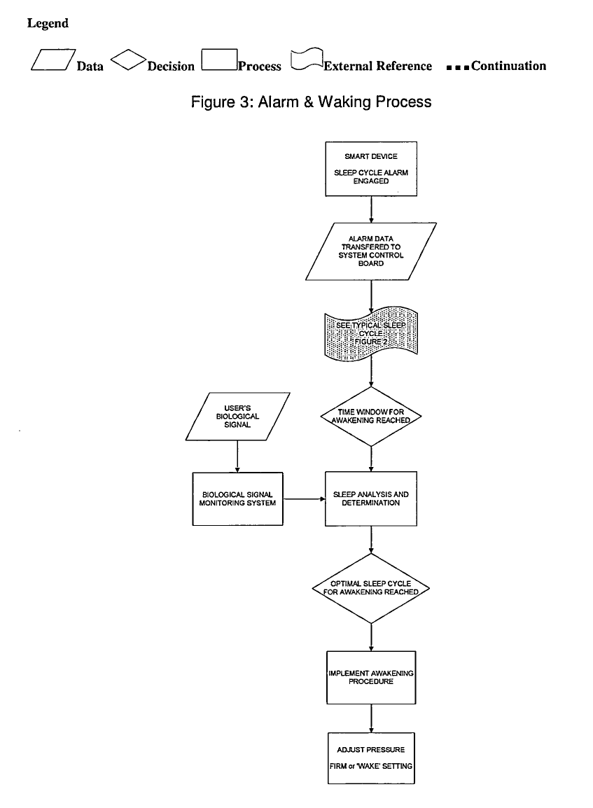

6: Bed as active part of waking a User
It is a claim of this art that it includes the first process that allows a bed to integrate a process specifically for the purpose of waking a User.
a) Direct control of a waking process as described in FIG. 3 is achieved through the nomination of a time by the User prior to using the bed and the automatic pressure adjustment to aid in the waking of that User at the defied time.
b) The integration of this active process aids in the boosting of the quality of the User's sleep through controlling the ending of the sleep in conjunction with the user's sleep pattern.

7: Waking Process
It is claim of this art that it will be the first process for the management of pressure to influence how a User is woken.
a) Expanding upon the existing available consumer Sleep Cycle technologies' that include Sleep Cycle monitoring as a key input into a User's awakening procedures. The Invention's processes utilize and coordinate physical stimulus through altering pressure as well as an alarm/sound emitted from a smart device for the purposes of waking the user.
b) FIG. 3 documents the process which includes the changing of pressure during a defined period prior, but reasonably associated with a preferred waking time.
c) The automation of the control in pressure is synchronized with the User's sleep pattern to bring the User to a ‘wakening’ or shallow sleep state.
d) The smart device or an associated piece of equipment will ultimately emit and alarm/sound within a defined period prior to the preferred waking time and coordinated with lifting the user to the aforementioned shallow sleep state.

8: Biological Signals
It is a claim of this art that a determinate of the User's Sleep Cycle will be a biological signal for the purposes of the operation of Invention.
a) It however is not a claim in this art that any particular biological signal over another should be preferred as an input into determining the User's sleep state
